(19) United States
(12) Patent Application Publication
(10) Pub. No.: US 2023/0404891 A1
(43) Pub. Date: Dec. 21, 2023

(54) COMPOSITION FOR SKINCARE

(71) Applicant: L'OREAL, Paris (FR)

(72) Inventors: Shan WU, Shanghai (CN); Chunyan HE, Shanghai (CN)

(73) Assignee: L'OREAL, Paris (FR)

(21) Appl. No.: 18/250,308

(22) PCT Filed: Oct. 30, 2020
(86) PCT No.: PCT/CN2020/125138

§ 371 Date: Apr. 24, 2023

(51) Int. Cl. A61K 8/67; A61Q 19/00; A61K 8/49; A61K 8/34

(52) U.S. Cl. CPC A61K 8/675; A61Q 19/00; A61K 8/498; A61K 8/345; A61K 8/342; A61K 2800/10

(57) ABSTRACT

A composition for skincare comprises in a hydrophilic phase: (i) at least one C5-C10 glycol; (ii) at least one linear unsaturated C18-C30 monoalcohol; and (iii) at least one hydrophilic cosmetic active ingredient. A non-therapeutic method for caring for the skin, comprising applying said composition to the skin, and use of the combination of at least one C5-C10 glycol and at least one linear unsaturated C18-C30 monoalcohol for improving the penetration of a hydrophilic cosmetic active ingredient into the skin.

### TECHNICAL FIELD

The present invention relates to a composition. In particular, the present invention relates to a composition for skincare. The present invention also relates to a non-therapeutic method for caring for the skin, and use of the combination of at least one C5-C10 glycol and at least one linear unsaturated C18-C30 monoalcohol for improving the penetration of a water soluble cosmetic active ingredient into the skin.

### BACKGROUND ART

The skin is the protective barrier for the human body. It protects the interior of the body from physical injury (such as trauma) and biological injury (such as bacteria, viruses or fungi). The skin of the human body comprises the dermis and the epidermis. The epidermis is the topmost layer of the skin, and its superficial layer is called the stratum corneum.

The development of formulations dedicated to caring for and/or making up the skin and/or lips, is permanent.

To date, some prior art documents relating to compositions comprising cosmetic active ingredients have been published.

JP-A-2011-73992 discloses a composition comprising the following components (A) to (F): (A) 10-50 parts by weight of one or more polyglycerin fatty acid esters with an HLB of 10-18, obtained from a polyglycerin and a fatty acid having 8 to 22 carbon atoms, (B) 1-30 parts by weight of one or more fatty acid monoglycerides obtained from glycerin and a fatty acid having 8 to 22 carbon atoms, (C) 0.1-30 parts by weight of an oil in the form of liquid at 25° C., (D) 10-35 parts by weight of a polyhydric alcohol, (E) 5-40 parts by weight of water, and (F) 0.01-10 parts by weight of a ceramide.

WO 2005/065630 A1 discloses a monophase micro-emulsion composition comprising (A) a hydrophilic nonionic surfactant, (B) a lipophilic nonionic surfactant, (C) oil, (D) an aqueous solvent immiscible with the oil, in which the critical micell concentration (c.m.c) of hydrophilic nonionic surfactant is higher than that in water, and (E) water.

WO 2004/045566 discloses a semitransparent cosmetic consisting of an O/W emulsion which comprises (a) a ceramide, (b) an oil component, (c) a nonionic surfactant, and (d) water and has a mean particle diameter of 100 to 300 nm.

However, for many compositions containing cosmetic active ingredient for caring for keratin materials, it is difficult for the active ingredient contained to penetrate into the keratin materials.

For compositions containing cosmetic active ingredients, penetration of the active ingredients to the stratum corneum is one of the most important properties.

There is thus still a need to formulate a composition for caring for the skin, which has an improved effect in terms of penetration of the cosmetic active ingredient contained into the stratum corneum.

### SUMMARY OF THE INVENTION

The inventors have now discovered that it is possible to formulate such compositions having an improved effect in terms of penetration of the cosmetic active ingredient contained to the stratum corneum.

Accordingly, in a first aspect, the present invention relates to a composition for skincare, comprising in a hydrophilic phase:

(i) at least one C5-C10 glycol;

(ii) at least one linear unsaturated C18-C30 monoalcohol; and

(iii) at least one hydrophilic cosmetic active ingredient.

The composition of the present invention can be in the form of an emulsion or a liquid, for example, a toner or a serum.

In a second aspect, the present invention relates to a non-therapeutic method for caring for the skin, comprising applying the composition according to the first aspect of the present invention to the skin.

It was found that the hydrophilic cosmetic active ingredient contained in the composition according to the present invention can easily penetrate into the stratum corneum.

In a third aspect, the present invention relates to use of the combination of at least one C5-C6 glycol and at least one linear unsaturated C18-C30 monoalcohol for improving the penetration of a hydrophilic cosmetic active ingredient into the skin.

Other subjects and characteristics, aspects and advantages of the present invention will emerge even more clearly from the detailed description and the examples that follow.

### BRIEF DESCRIPTION OF THE DRAWINGS

Implementations of the present invention will now be described, by way of example only, with reference to the attached figures, wherein:

FIG. 1 shows the Raman spectra of 400-2000cm⁻1 for niacinamide.

FIG. 2 shows the Raman spectra of 400-2000cm⁻1 for proxylane.

FIG. 3 shows the penetration profile of niacinamide and proxylane for the composition of background 1.

FIG. 4 shows the penetration profile of niacinamide and proxylane for the composition of background 2.

FIG. 5 shows the penetration profile of niacinamide and proxylane for the composition of comparative formula 1;

FIG. 6 shows the penetration profile of niacinamide and proxylane for the composition of comparative formula 2;

FIG. 7 shows the penetration profile of niacinamide and proxylane for the composition of comparative formula 3;

FIG. 8 shows the penetration profile of niacinamide and proxylane for the composition of invention formula 1;

FIG. 9 shows a comparison of penetration profile of niacinamide for the compositions of comparative formulas 1-3 and invention formula 1; and

FIG. 10 shows a comparison of penetration profile of proxylane for the compositions of comparative formulas 1-3 and invention formula 1.

### DETAILED DESCRIPTION OF THE INVENTION

According to the first aspect, the present invention provides a composition for skincare, comprising, in a hydrophilic phase:

(i) at least one C5-C10 glycol;

(ii) at least one linear unsaturated C18-C30 monoalcohol; and

(iii) at least one hydrophilic cosmetic active ingredient.

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “ranging from . . . to . . .”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

All percentages in the present invention refer to weight percentage, unless otherwise specified.

### Hydrophilic Phase

According to the first aspect, the composition of the present invention comprises a hydrophilic phase.

The hydrophilic phase comprises at least one solvent selected from water and C2-C4 glycol.

Preferably, the hydrophilic phase comprises at least one organic solvent miscible with water (at room temperature 25° C.) such as for example monoalcohols having from 2 to 6 carbon atoms such as ethanol, isopropanol, triols such as glycerin.

In some preferred embodiments, the hydrophilic phase of the composition of the present invention comprises water and glycerin.

If presents, water is preferably present in the composition of the present invention in an amount ranging from 1% to 80% by weight, preferably from 5% to 77% by weight, more preferably from 10% to 75% by weight, relative to the total weight of the composition.

Said hydrophilic phase is preferably present in an amount ranging from 10% to 99% by weight, more preferably from 20% to 90% by weight, and even more preferably from 50% to 85% by weight of the total weight of the composition.

### C5-C10 Glycol

According to the first aspect, the composition of the present invention comprises at least one C5-C10 glycol.

Preferably, the glycol has 5-8 carbon atoms, i.e, being C5-C8 glycol.

More preferably, the glycol is selected from C5-C6 glycol.

Thus, in some preferred embodiments, the composition of the present invention comprises at least one C5-C8 glycol, preferably C5-C6 glycol.

As examples of C5-C10 glycols that can be used in the composition of the present invention, mention can be made to pentylene glycol, hexylene glycol, dipropylene glycol, heptanediol, octanediol, nonanediol, and decylene glycol.

In the present invention, the definition of glycols includes all possible isomers. For example, pentylene glycol comprises 1,5-pentylene glycol, 2,4-pentylene glycol, etc.

In a more preferred embodiment, the composition comprises pentylene glycol.

Advantageously, the C5-C10 glycol is present in an amount ranging from 0.1% to 10%, preferably from 0.2% to 5% by weight, more preferably from 0.5% to 3% by weight, relative to the total weight of the composition.

Linear Unsaturated C18-C30 Monoalcohol

According to the first aspect, the composition of the present invention comprises at least one linear unsaturated C18-C30 monoalcohol.

The linear unsaturated C18-C30 monoalcohol is of structure R-OH with R denoting a linear alkenyl group comprising from 18 to 30 carbon atoms.

Preferably, the linear unsaturated C18-C30 monoalcohol is selected from monoalcohol of structure R-OH with R denoting a linear alkenyl comprising from 18 to 24 carbon atoms.

Most preferably, the linear unsaturated C18-C30 monoalcohol is oleyl alcohol.

As commerical products of linear unsaturated C18-C30 monoalcohol, mention can be made of oleyl alcohol sold under the name HD OCENOL 80/85 V/MB by BASF or CRODACOL A 10 by CRODA.

Advantageously, the linear unsaturated C18-C30 monoalcohol is present in an amount ranging from 0.1% to 10%, preferably from 1% to 10% by weight, more preferably from 2% to 8% by weight, relative to the total weight of the composition.

### Hydrophilic Cosmetic Active Ingredients

According to the first aspect, the composition of the present invention comprises at least one hydrophilic cosmetic active ingredient.

For the purpose of the present invention, the term “hydrophilic cosmetic active ingredient” means cosmetic active ingredient soluble or disperable in the hydrophilic phase defined above.

As examples of hydrophilic cosmetic active ingredient, mention can be made of:

terephthalylidene dicamphor sulfonic acid (Ecamsule), phenylbenzimidazole sulfonic acid (Ensulizole), Benzophenone-4, aminobenzoic acid (PABA), camphor benzalkonium methosulfate, methylene bis-benzotriazolyl tetramethylbutylphenol (Bisoctrizole), disodium phenyl dibenzimidazole tetrasulfonate (Bisdisulizole disodium), tris-biphenyl triazine; their derivatives and corresponding salts; naphthaline bisimide derivatives, and cinnamido amine cationic quaternary salts and derivatives, and mixtures thereof;

proxylane(hydroxypropyl tetrahydropyrantriol), flavones, stilbenoids, tannins, phenolic acids, polyphenolics, vitamins, xanthines, ceramides, cholesterols, sphingosines, C-glycosides, zwitterionic N-substituted amino sulfonic acid buffers, sugars, nucleic acids, a- and β-hydroxy acids, aminopropyl triethoxysilane, dihydroxyacetone, botanical extracts, amino acids, and peptides, their derivatives, and combinations thereof; and

ascorbic acid and its biologically compatible salts, enzymes, antibiotics, components having a tautening effect, .alpha.-hydroxy acids and their salts, hydroxylated polyacids, sucroses and their derivatives, urea, amino acids, oligopeptides, carnosine, acetyl tetrapeptide-9, palmitoyl tripeptide-1, water-soluble plant and yeast extracts, protein hydrolysates, hyaluronic acid, mucopolysaccharides, vitamins B 2 , B 3 (niacinamide), B₆, H and PP, panthenol, folic acid, acetylsalicylic acid, allantoin, glycyrrhetic acid, kojic acid and hydroquinone.

Preferably, the hydrophilic cosmetic active ingredient is selected from proxylane (hydroxypropyl tetrahydropyrantriol), niacinamide, and a mixture thereof.

Advantageously, the hydrophilic cosmetic active ingredient is present in an amount ranging from 0.1% to 40% by weight, preferably from 1 to 35% by weight, more preferably from 10% to 35% by weight, relative to the total weight of the composition.

### Fatty Phase

In some embodiments, the composition of the present invention further comprises a fatty phase.

Said fatty phase preferably comprises at least one oil. The oil can be volatile or non-volatile.

The term “oil” means a water-immiscible non-aqueous compound that is liquid at room temperature (25° C.) and at atmospheric pressure (760 mmHg).

The term “non-volatile oil” means an oil that may remain on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Preferably, said oil is selected from hydrocarbonated, silicone or fluorinated oils.

The term “hydrocarbon-based oil” or “hydrocarbonated oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

The term “silicone oil” means an oil containing at least one silicon atom, especially containing Si-O groups.

The term “fluorinated oil” means an oil containing at least one fluorine atom.

The fatty phase can be, for example, present in an amount ranging from 0.01% to 50% by weight, preferably from 0.05% to 30% by weight, more preferably from 0.1% to 10% by weight, relative to the total weight of the composition.

### Additional Adjuvants or Additives

The composition of the present invention may comprise conventional cosmetic adjuvants or additives, for instance fragrances, chelating agents (for example, disodium EDTA), preserving agents (for example, chlorphenesin and phenoxyethanol) and bactericides, surfactants, thickeners, fillers, pH regulators (for example citric acid, sodium hydroxide, potassium hydroxide), and mixtures thereof.

According to a particularly preferred embodiment, the present invention provides a composition for skincare, comprising in a hydrophilic phase, relative to the total weight of the composition:
(i) from 0.5% to 3% by weight of pentylene glycol;
(ii) from 2% to 8% by weight of oleyl alcohol; and
(iii) from 10% to 35% by weight of at least one hydrophilic cosmetic active ingredient selected from proxylane, niacinamide, and a mixture thereof.

### Method and Use

According to the second aspect, the present invention relates to a non-therapeutic method for caring for the skin, comprising applying the composition according to the first aspect of the present invention to the skin.

In particular, the keratin material is the skin.

Thus, in an embodiment of the second aspect, the present invention provides a non-therapeutic method for caring for the skin, comprising applying the composition according to the first aspect of the present invention to the skin.

In a third aspect, the present invention relates to use of the combination of at least one C5-C10 glycol and at least one linear unsaturated C18-C30 monoalcohol for improving the penetration of a hydrophilic cosmetic active ingredient into the skin.

The C5-C10 glycol and the linear unsaturated C18-C30 monoalcohol are defined as above.

Preferably, the at least one C5-C10 glycol is selected from C5-C8 glycol, and the at least one linear unsaturated C18-C30 monoalcohol is selected from monoalcohol of structure R-OH with R denoting a linear alkenyl comprising from 18 to 24 carbon atoms.

More preferably, the at least one C5-C10 glycol is pentylene glycol, and the at least one linear unsaturated C18-C30 monoalcohol is oleyl oil.

Preferably, the hydrophilic cosmetic active ingredient is selected from proxylane, niacinamide, and a mixture thereof.

The following examples serve to illustrate the present invention without, however, being limiting in nature.

### EXAMPLES

### Example 1: Preparation of Compositions

Compositions according to comparative formulas (Comp.) and invention formula (Inv.) were prepared according to the contents given in Table 1 (the contents are expressed as weight percentages of active material relative to the total weight of each composition, unless otherwise indicated).

TABLE 1

| | Comp. 1 | Comp. 2 | Comp. 3 | Inv. 1 |
|---|---|---|---|---|
| INCI US(Commercial name and supplier) | Wt. % | Wt. % | Wt. % | Wt. % |
| PHENOXYETHANOL | 0.50 | 0.50 | 0.50 | 0.50 |
| POTASSIUM HYDROXIDE | 0.15 | 0.15 | 0.15 | 0.15 |
| GLYCERIN | 5.25 | 5.25 | 5.25 | 5.25 |
| SODIUM POLYACRYLATE(COVACRYL | 0.80 | 0.80 | 0.80 | 0.80 |
| MV60 from SENSIENT) | | | | |
| XANTHAN GUM (KELTROL ® CG-T from | 0.26 | 0.26 | 0.26 | 0.26 |
| CP KELCO) | | | | |
| STEARIC ACID | 7.00 | 7.00 | 7.00 | 7.00 |
| GLYCERYL STEARATE (and) PEG-100 | 0.80 | 0.80 | 0.80 | 0.80 |
| STEARATE(SP ARLACEL 165 FP-SEG-PA- | | | | |
| (RB) from CRODA) | | | | |
| CAPRYLIC/CAPRIC | 3.50 | 3.50 | 3.50 | 3.50 |
| TRIGLYCERIDE(MASESTER ™ E7000 LR01- | | | | |
| 1/MB from PT MUSIM MAS) | | | | |
| PENTAERYTHRITYL | 1.70 | 1.70 | 1.70 | 1.70 |
| TETRAISOSTEARATE(CRODAMOL ™ PTIS- | | | | |
| LQ-(MH) from CRODA) | | | | |
| ISONONYL ISONONANOATE(ERCAREL | 1.70 | 1.70 | 1.70 | 1.70 |
| ISN/O from ERCA) | | | | |
| BUTYROSPERMUM PARKII (SHEA) | 2.60 | 2.60 | 2.60 | 2.60 |
| BUTTER(FAIR FOR LIFE REFINED SHEA | | | | |
| BUTTER from OLVEA) | | | | |
| CAPRYLYL METHICONE(DOW CORNING | 1.70 | 1.70 | 1.70 | 1.70 |
| FZ-3196 from DOW CORNING) | | | | |
| BIS-PEG/PPG-16/16 PEG/PPG-16/16 | 0.90 | 0.90 | 0.90 | 0.90 |
| DIMETHICONE (and) CAPRYLIC/CAPRIC | | | | |
| TRIGLYCERIDE(ABIL ® CARE 85 from | | | | |
| EVONIK GOLDSCHMIDT) | | | | |
| MYRISTYL MYRISTATE(RADIA 7744 RSPO | 0.90 | 0.90 | 0.90 | 0.90 |
| MASS BALANCE from OLEON) | | | | |
| WATER | QS100 | QS100 | QS100 | QS100 |
| HYDROXYPROPYL | 30.00 | 30.00 | 30.00 | 30.00 |
| TETRAHYDROPYRANTRIOL(MEXORYL | | | | |
| SCN from CHIMEX (NOVEAL)) | | | | |
| NIACINAMIDE(NIACINAMIDE PC from | 1.00 | 1.00 | 1.00 | 1.00 |
| DSM NUTRITIONAL PRODUCTS) | | | | |
| PENTYLENE GLYCOL | — | — | 3.50 | 1.50 |
| PROPYLENE GLYCOL | — | 1.5 | — | |
| OLEYL ALCOHOL | — | 2.00 | — | 2.00 |

Composition of comparative formula 1 does not comprise C5-C10 glycol and linear unsaturated C18-C30 monoalcohol.

Composition of comparative formula 2 does not comprise C5-C10 glycol.

Composition of comparative formula 3 does not comprise linear unsaturated C18-C30 monoalcohol.

The following two compositions were also prepared as baseline compositions for baselines for Raman spectroscopy test described below according to the contents given in Table 2 (the contents are expressed as weight percentages of active material relative to the total weight of each composition, unless otherwise indicated).

TABLE 2

| INCI US(Commercial name | Background 1 | Background 2 |
|---|---|---|
| and supplier) | Wt. % | Wt. % |
| PHENOXYETHANOL | 0.50 | 0.50 |
| POTASSIUM HYDROXIDE | 0.15 | 0.15 |
| GLYCERIN | 5.25 | 5.25 |
| SODIUM POLYACRYLATE | 0.80 | 0.80 |
| XANTHAN GUM | 0.26 | 0.26 |
| STEARIC ACID | 7.00 | 7.00 |
| GLYCERYL STEARATE (and) | 0.80 | 0.80 |
| PEG-100 STEARATE | | |
| CAPRYLIC/CAPRIC | 3.50 | 3.50 |
| TRIGLYCERIDE | | |
| PENTAERYTHRITYL | 1.70 | 1.70 |
| TETRAISOSTEARATE | | |
| ISONONYL ISONONANOATE | 1.70 | 1.70 |
| BUTYROSPERMUM PARKII | 2.60 | 2.60 |
| (SHEA) BUTTER | | |
| CAPRYLYL METHICONE | 1.70 | 1.70 |
| BIS-PEG/PPG-16/16 | 0.90 | 0.90 |
| PEG/PPG-16/16 | | |
| DIMETHICONE (and) | | |
| CAPRYLIC/CAPRIC | | |
| TRIGLYCERIDE | | |
| MYRISTYL MYRISTATE | 0.90 | 0.90 |
| WATER | QS100 | QS100 |
| PENTYLENE GLYCOL | — | 1.50 |
| OLEYL ALCOHOL | — | 2.00 |

### Preparation Process

The compositions listed above were prepared as follows, taking the composition of invention formula 1 as an example:

1. Mixing water, niacinamide, hydroxypropyl tetrahydropyrantriol(proxylane), pentylene glycol, glycerin, phenoxyethanol, potassium hydroxide to obtain a hydrophilic phase, and heating the hydrophilic phase to 75° C.;

2. Mixing stearic acid, glyceryl stearate (and) PEG-100 stearate, caprylic/capric triglyceride, pentaerythrityl tetraisostearate, isononyl isononanoate, butyrospermum parkii (rhea) butter, caprylyl methicone, bis-PEG/PPG-16/16 PEG/PPG-16/16 dimethicone (and) caprylic/capric triglyceride; myristyl myristate, and oleyl alcohol to obtain an oil phase, and heating the oil phase to 85° C.;

3. Adding the oil phase into the hydrophilic phase, and homogenizing at 1500-2000 rpm for 10 minutes to obtain a mixture;

4. Adding sodium polyacrylate and xanthan gum into the mixture;

5. Cooling the mixture to 55° C., and adding fragrance (if needed), and cooling to room temperature to obtain the composition.

### Example 2: Evaluation of compositions

The penetration of cosmetic active ingredients in each composition prepared in Example 1 was characterized as follows.

### i) Preparation of Skin Tissue Samples

6 μl of composition of each formula was applied evenly on 0.8cm×0.8 cm porcine skin (pig ear skin from food industry), corresponding to 9 mg/cm². The porcine skin sample was then emerged on insert membrane with PBS underneath, followed by 37° C. incubation at 95% RH for 2 hours. Treated sample was embedded in an OCT Tissue Freezing Medium, then frozen and cryo-sectioned into 20 μm thickness. It was further placed on a CaF2 substrate for Raman confocal scanning. Three porcine samples were prepared for each formula. A Raman confocal mapping was acquired of each treated sample.

### ii) Raman Spectroscopy

A LabRam HR Evolution (Horiba Jobin-Yvon, Villeneuve-d′Ascq, France) Raman confocal microscope was used. Raman spectrum was obtained using a 532 nm DPSS laser with a power of 8 mW on the sample, coupled with a ×50 LM Plan objective (Olympus, NA 0.75 Rungis, France). The confocal hole was set at 100 μm diameter for all measurements. The system was spectrally calibrated to the 520.7 cm⁻1 spectral line of silicon before the test. Detection was facilitated by dispersing Raman-shifted radiation onto a charge-coupled device (CCD) detector using a grating of 600 lines/mm.

For pure cosmetic active ingredients, single point spectra were acquired with 25% laser intensity and 10 seconds acquisition time, for 400-2000cm⁻¹ spectral range.

For mapping in the sample evaluation, the step size was 3 μm in both X and Y direction. The acquisition areas were 18×150 μm. For each spot, 50% laser intensity and 5 seconds acquisition time per spectrum was used. Spectral range was 400-2000 cm⁻⁻1.

### iii) Data Analysis

Non-negative constrained least square (NCLS) analysis was performed using Matlab. Before statistical analysis, Raman spectra were subjected to a linear baseline correction. Cosmetic active ingredient spectral of 400-2000cm⁻1 fingerprints were used for analysis.

FIG. 1 shows the Raman spectra of 400-2000cm⁻¹ for niacinamide.

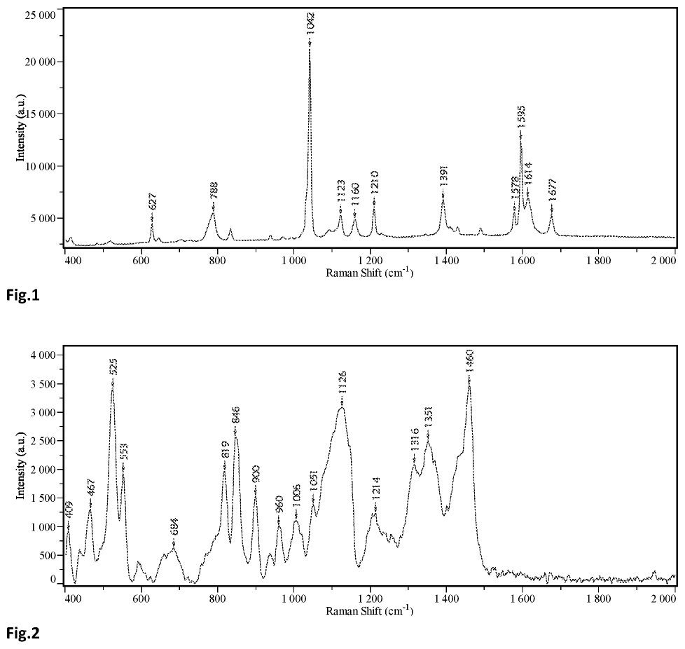

FIG. 2 shows the Raman spectra of 400-2000cm⁻1 for proxylane.

A simplified description of the NCLS outcome can be defined as:

Ss=(SR1*C1)+(SR2*C2)+. . . +(SRi*Ci)+R*CR

Ss: Acquired Raman signal of one pixel on treated porcine skin sample;

SRi: Raman signal of each suppositional ingredient (PCA component) in untreated porcine;

R: Raman signal of pure cosmetic active ingredient;

Ci: coefficient of each suppositional ingredient (PCA component) in the exact pixel;

CR: coefficient of cosmetic active ingredient in the exact pixel;

The computational co-efficient index of active ingredients can be used to generate the distribution profile of ACIs from outer skin stratum corneum to the deeper dermis part.

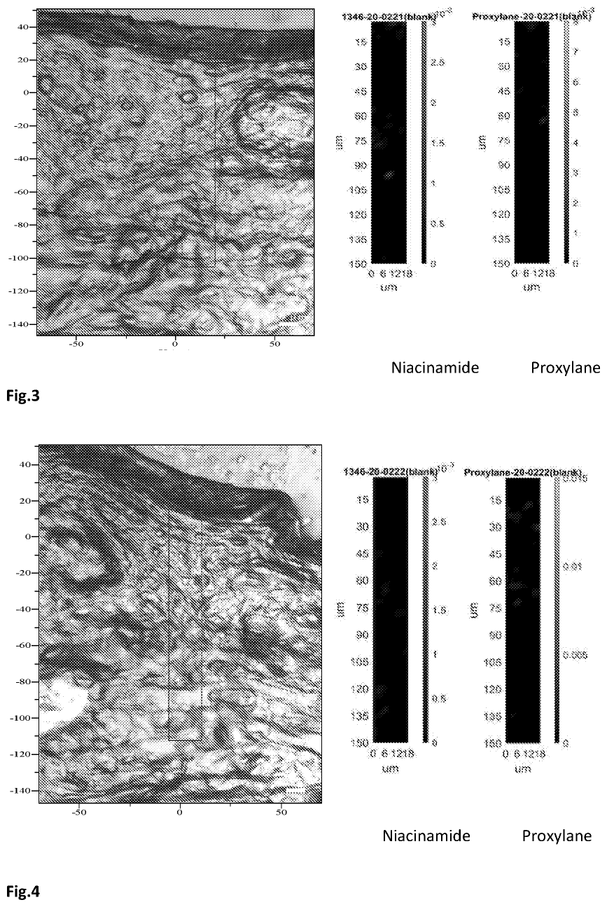

FIG. 3 shows the penetration profile of niacinamide and proxylane for the composition of background 1.

FIG. 4 shows the penetration profile of niacinamide and proxylane for the composition of background 2.

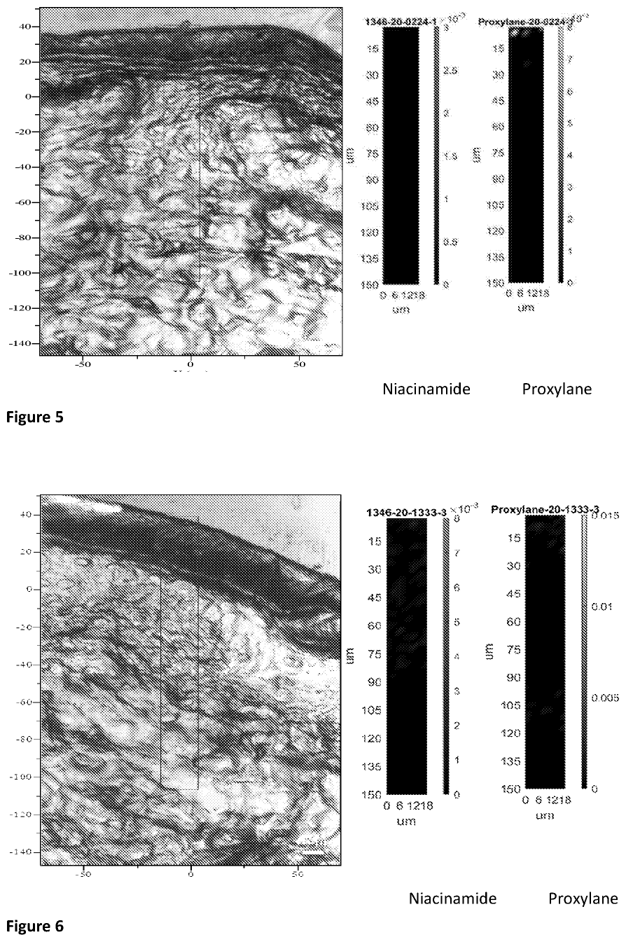

FIG. 5 shows the penetration profile of niacinamide and proxylane for the composition of comparative formula 1.

FIG. 6 shows the penetration profile of niacinamide and proxylane for the composition of comparative formula 2;

FIG. 7 shows the penetration profile of niacinamide and proxylane for the composition of comparative formula 3;

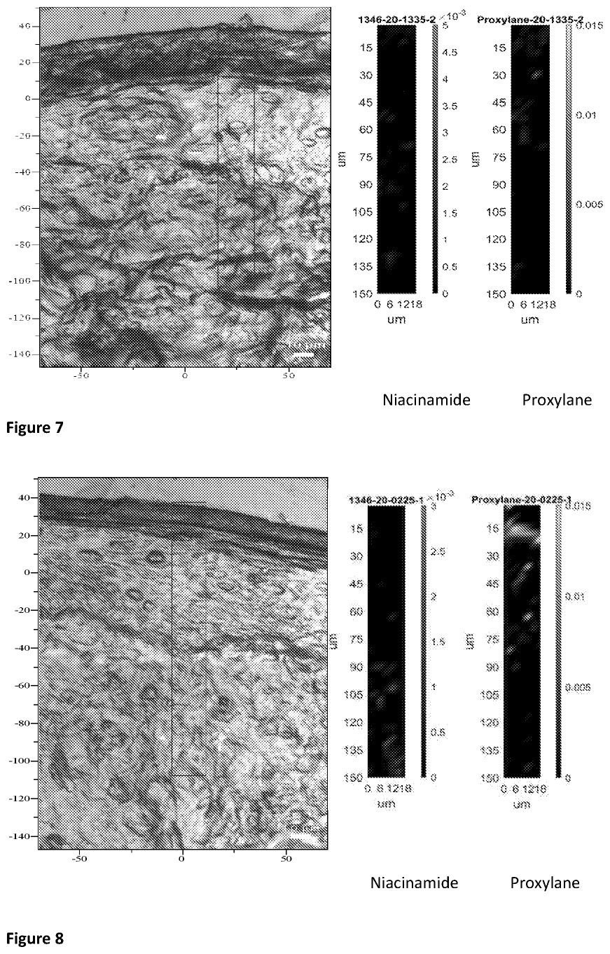

FIG. 8 shows the penetration profile of niacinamide and proxylane for the composition of invention formula 1;

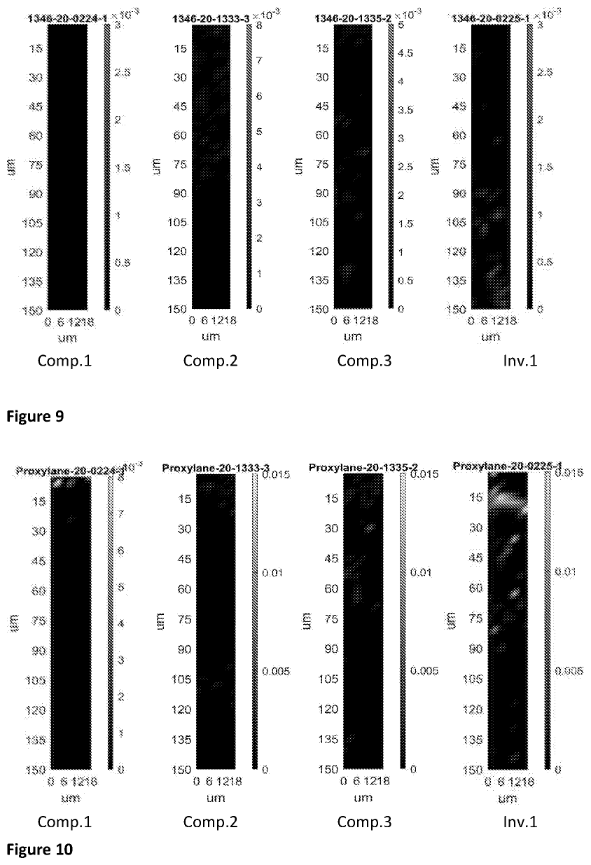

FIG. 9 shows a comparison of penetration profile of niacinamide for the compositions of comparative formulas 1-3 and invention formula 1; and

FIG. 10 shows a comparison of penetration profile of proxylane for the compositions of comparative formulas 1-3 and invention formula 1.

It can be seen from FIG. 9 that the combination of C5-C10 glycol and linear unsaturated C18-C30 monoalcohol can improve the penetration of niacinamide into the skin after topical application.

It can be seen from FIG. 10 that the combination of C5-C10 polyol and linear unsaturated C18-C30 monoalcohol can improve the penetration of proxylane into the skin after topical application.

1. A composition for skincare, comprising in a hydrophilic phase:
(i) at least one C5-C10 glycol;
(ii) at least one linear unsaturated C18-C30 monoalcohol; and
(iii) at least one hydrophilic cosmetic active ingredient.

2. The composition according to claim 1, wherein the C5-C10 glycol is selected from the group consisting of pentylene glycol, hexylene glycol, dipropylene glycol, heptanediol, octanediol, nonanediol, and decylene glycol.

3. The composition according to claim 1, wherein the C5-C10 glycol is present in an amount ranging from 0.1% to 10%, relative to the total weight of the composition.

4. The composition according to claim 1, wherein the linear unsaturated C18-C30 monoalcohol is of structure R-OH with R denoting a linear alkenyl group comprising from 18 to 30 carbon atoms.

5. The composition according to claim 1, wherein the linear unsaturated C18-C30 monoalcohol is selected from linear monoalcohols having from 18 to 24 carbon atoms.

6. The composition according to claim 1, wherein the linear unsaturated C18-C30 monoalcohol is present in an amount ranging from 0.1% to 10% relative to the total weight of the composition.

7. The composition according to claim 1, wherein the hydrophilic cosmetic active ingredient is present in an amount ranging from 0.1% to 40% relative to the total weight of the composition.

8. The composition according to claim 1, further comprising conventional cosmetic adjuvants or additives selected from the group consisting of fragrances, chelating agents, preserving agents, bactericides, surfactants, thickeners, fillers, pH regulators, and mixtures thereof.

9. The composition according to claim 1, comprising in a hydrophilic phase, relative to the total weight of the composition:
(i) from 0.5% to 3% by weight of pentylene glycol;
(ii) from 2% to 8% by weight of oleyl alcohol; and
(iii) from 10% to 35% by weight of at least one hydrophilic cosmetic active ingredient selected from proxylane, niacinamide, and a mixture thereof.

10. The composition according to claim 1, wherein the hydrophilic phase comprises at least one solvent selected from the group consisting of water and C2-C4 glycol.

11. A non-therapeutic method for caring for the skin, comprising applying the composition according to claim 1.

12. A method to improve penetration of a hydrophilic cosmetic active ingredient into the skin, comprising adding a combination of at least one C5-C10 glycol and at least one linear unsaturated C18-C30 monoalcohol to a hydrophilic cosmetic active ingredient into the skin.

13. The method according to claim 12, wherein the at least one C5-C10 glycol is selected from the group consisting of C5-C8 glycols gel, and the at least one linear unsaturated C18-C30 monoalcohol is selected from monoalcohols of structure R-OH with R denoting a linear alkenyl comprising from 18 to 24 carbon atoms.

14. The method according to claim 12, wherein the at least one C5-C10 glycol is pentylene glycol, and the at least one linear unsaturated C18-C30 monoalcohol is oleyl oil.

15. The method according to claim 13, wherein the hydrophilic cosmetic active ingredient is selected from the group consisting of proxylane, niacinamide, and a mixture thereof.
